(19) United States
(12) Patent Application Publication
(10) Pub. No.: US 2017/0172796 A1
(43) Pub. Date: Jun. 22, 2017

(54) SURGICAL SYSTEM WITH SUBSTANCE DELIVERY SYSTEM

(71) Applicant: Novartis AG, Basel (CH)

(72) Inventors: Philip John Biancalana, Keller, TX (US); Mark Alan Hopkins, Mission Viejo, CA (US); Michael James Papac, North Tustin, CA (US); Robert Joseph Sanchez, JR., Oceanside, CA (US)

(21) Appl. No.: 15/378,441

(22) Filed: Dec. 14, 2016

Related U.S. Application Data
(60) Provisional application No. 62/268,001, filed on Dec. 16, 2015.

(51) Int. Cl. A61F 9/007; A61M 5/00; A61M 39/24

(52) U.S. Cl. CPC A61F 9/00754; A61M 39/24; A61M 5/007; A61B 2217/005; A61B 2217/007; A61M 2039/226

(57) ABSTRACT

A microsurgical system with an integrated substance chamber is provided herein for delivering substance (e.g., a drug, retina patch, dye, etc.) to a surgical site through a tool component of the microsurgical instrument, such as a tissue removal component. The system may include a tissue removal handpiece having a tissue cutter disposed at a distal end. The system may further include a first fluid conduit and a second fluid conduit. The first fluid conduit may couple a fluidics subsystem to the tissue removal handpiece to permit removal of cut tissue. The second fluid conduit may couple a substance chamber to the tissue removal handpiece. The system may additionally include a control system that enables or facilitates selective control of fluid through the tissue removal handpiece to introduce substance from the substance chamber to a surgical site through a port in the tissue cutter.

### TECHNICAL FIELD

The present disclosure is directed to methods and systems for medical procedures, and more particularly, to methods and systems involving a need for delivering a substance into tissues within a body cavity.

### BACKGROUND

Vitreo-retinal procedures are commonly performed to treat many serious conditions of the posterior segment of the eye. For example, vitreo-retinal procedures may treat conditions such as age-related macular degeneration (AMD), diabetic retinopathy and diabetic vitreous hemorrhage, macular hole, retinal detachment, epiretinal membrane, cytomegalovirus (CMV) retinitis, and many other ophthalmic conditions.

When performing vitreo-retinal procedures, a surgeon may use a microscope with special lenses designed to provide a clear image of the posterior segment of the eye. As access points, the surgeon may make several tiny incisions just a millimeter or so in diameter on the sclera at the pars plana. The surgeon inserts microsurgical instruments through the incisions, such as a fiber optic light source to illuminate inside the eye, an infusion conduit to maintain the eye's shape during surgery, and instruments to cut and remove the vitreous body. Conventionally, a separate incision may be provided for each microsurgical instrument when using multiple instruments simultaneously.

During such surgical procedures, proper illumination and visualization of the tissue to be treated or removed, such as vitreous, is important. Some vitreoretinal procedures require removal of the vitreous from the posterior segment of the eye. Because the vitreous is clear, visualization can be unusually difficult compared with some other types of tissues. In order to better visualize the clear vitreous, a dye may be introduced into the cavity of the eye. The dye adheres to the vitreous, which can then be seen more easily and, consequently, can be removed more easily.

In some instances, the dye (or another substance such as a drug or retina patch) may be introduced into the eye through an access cannula extending through the sclera. However, this may require either the removal of one of the tools being used in a given procedure or an additional incision in the eye. Either of these options results in increased surgical time and potential complications. Additionally, when a tool is removed and replaced after injection of the substance, if additional substance is needed (e.g., to better visualize remaining vitreous or to provide additional drug volume), the process of removal, injection, and replacement may need to be repeated.

### SUMMARY

The present disclosure is directed to exemplary microsurgical systems that are configured to inject a substance into a surgical site. The systems may include a tissue removal surgical system having microsurgical instrument and a substance chamber for delivering a substance to a surgical site through a tool component of the microsurgical instrument, such as a tissue removal component. An exemplary system may include a tissue removal handpiece having a tissue cutter disposed at a distal end. The system may further include a first fluid conduit and a second fluid conduit. The first fluid conduit may couple a fluidics subsystem to the tissue removal handpiece to permit removal of cut tissue. The second fluid conduit may couple a substance chamber to the tissue removal handpiece. The system may additionally include a control system that enables or facilitates selective control of fluid through the tissue removal handpiece to introduce a substance from the substance chamber to a surgical site through a port in the tissue cutter. For example, the control system may include a switch, button, slider, or roller on an exterior of the handpiece and accessible to the user for controlling the introduction of the substance from the substance chamber to the exterior of the handpiece.

Exemplary surgical instruments are provided herein. An exemplary surgical instrument for use in an ophthalmic procedure may include an elongate tubular member having a distal end for insertion through eye tissue into the vitreous chamber of an eye. The elongate tubular member may have an inner lumen extending therethrough, and a portion of the elongate tubular member may extend within a housing. The surgical instrument may include a first fluid conduit to couple the housing to a fluidics subsystem in order to aspirate vitreous from the vitreous chamber of the eye. The first fluid conduit may be in fluid communication with the lumen of the elongate tubular member. The surgical instrument may also include a substance chamber coupled to the first fluid conduit to provide a substance into the vitreous chamber of the eye. The substance chamber may be coupled to the first fluid conduit at a first connection site via a second fluid conduit and at a second connection site via a third fluid conduit, in some embodiments. The surgical instrument may include a plurality of valves. For instance, a first valve may be positioned along the second fluid conduit and a second valve may be positioned along the first fluid conduit between the first connection site and the second connection site.

Exemplary vitrectomy systems are provided herein. An exemplary vitrectomy system may include a handpiece having a vitrectomy cutter and a fluid conduit connecting to the handpiece and coupling to the vitrectomy cutter. The vitrectomy system may further include a housing configured for use in a user's hand during a vitrectomy procedure and a vitrectomy cutter protruding from a distal end of the housing. The vitrectomy cutter may include an inner cutting tube having an inner cutting port at a distal tip thereof and an elongate tubular member having a lumen extending therethrough. The inner cutting tube may extend within the lumen of the elongate tubular member. The vitrectomy cutter may further include an outer cutting tube having an outer port. The outer cutting tube may extend from a distal end of the elongate tubular member. The vitrectomy system may further include a fluid line coupled to the inner cutting tube and a substance infusion conduit coupled to the handpiece and coupled to the elongate tubular member.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the accompanying drawings and the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 illustrates a perspective view of an exemplary surgical system, according to an embodiment consistent with the principles of the present disclosure.

FIG. 2 is an illustration of an exemplary block diagram of the surgical system of FIG. 1, according to an aspect consistent with the principles of the present disclosure.

FIG. 3 is a cross-sectional illustration of an exemplary surgical tool, according to aspects of the present disclosure.

FIG. 4 is an illustration of an exemplary tissue removal system including a vitrectomy probe configured to facilitate substance injection, according to aspects of the present disclosure.

FIG. 5 is an illustration of an exemplary tissue removal system including a vitrectomy probe configured to facilitate substance injection, according to aspects of the present disclosure.

FIG. 6 is an illustration of an exemplary tissue removal system including a vitrectomy probe configured to facilitate substance injection, according to aspects of the present disclosure.

FIG. 7A is top-view illustration of an exemplary vitrectomy probe configured to facilitate substance injection, according to aspects of the present disclosure.

FIG. 7B is cross-sectional illustration of the exemplary vitrectomy probe of FIG. 7A as seen along line A-A, according to aspects of the present disclosure.

FIG. 7C is an off-axis perspective of the exemplary vitrectomy probe of FIG. 7A, according to aspects of the present disclosure.

FIG. 7D is a detailed perspective view showing a distal tip of the vitrectomy probe of FIG. 7A, according to aspects of the present disclosure.

FIG. 8 is a method for removing body tissue from a body cavity using an integrated surgical tool, according to aspects of the present disclosure.

The accompanying drawings may be better understood by reference to the following detailed description.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

The present disclosure is directed to methods and systems for injecting a substance (e.g., a drug, retina patch, visualization dye, etc.) into a body cavity during an operation without requiring a separate incision to be made and without requiring introduction of a separate cannula into the body cavity. In some examples, a substance may be injected into the cavity through a lumen of an elongate portion of a tissue removal surgical tool, such as a vitrectomy probe. For example, a user may perform a procedure that includes removal of vitreous from the eye using a vitrectomy probe. The same vitrectomy probe may also introduce a substance into the eye. Accordingly, a surgeon or other user may be able to cut and remove vitreous and inject an amount of the substance into the vitreous chamber of the eye of the patient using the same tool. The substance may include a drug (for example, but not limited to, a vitreous dissolution drug). Other drugs are also contemplated. The substance may also be a retina patch. As another example, the substance may be or may include a dye. If the substance is a dye, removal of vitreous may be simplified by allowing the dye to be more easily introduced, increasing the ability to visualize the vitreous during the removal procedure. This may also reduce the incidence of post-operative complications, including, for example, post-operative retinal tearing, retinal detachment, and other complications. Other substances are also contemplated.

Depending on the implementation, the vitreous cutter may be coupled to or may include a substance chamber. In implementations where the vitreous probe is coupled to the substance chamber, an aspiration line may extend between the vitreous probe and a surgical console. Using controls locating on the vitreous probe and/or the surgical console (such as a footpedal connected thereto), the surgeon may inject a portion of the substance from the substance chamber through a portion of the aspiration line during a reflux process. Other examples of controls include a switch, button, slider, or roller on an exterior of the handpiece and accessible to the user for controlling the injection of the substance. As used herein, a reflux process refers to a reversal in the direction of fluid flow. During an aspiration process, the vitreous probe may aspirate vitreous from the eye as in a conventional vitreous removal process. Embodiments of the present disclosure may enable a surgeon to use a reflux process to controllably inject a desired amount of the substance into the surgical site without removing the vitreous cutter from the site. In the case of a dye, visualization may be improved during a vitreous removal procedure to increase procedure efficiency and may provide better patient outcome. Other implementations do not use a reflux process, but inject the substance without changing direction of the aspiration fluid flow.

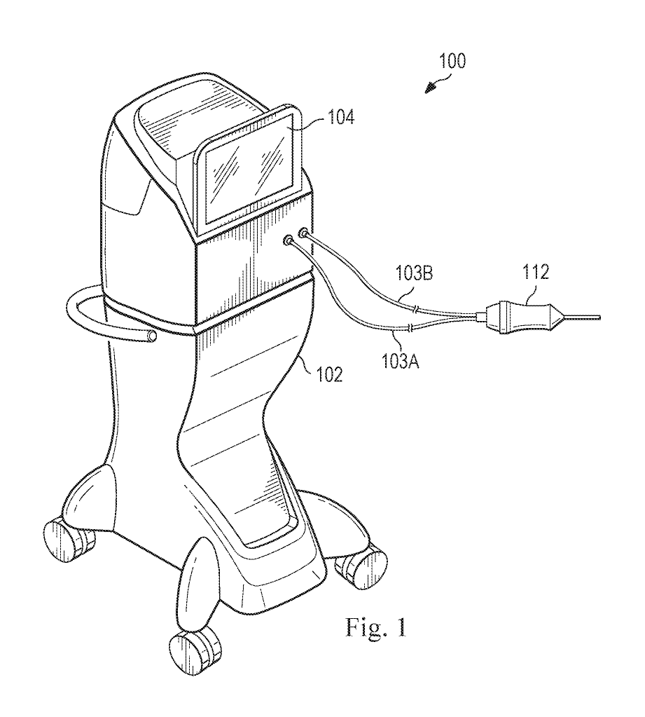

FIG. 1 illustrates a tissue removal surgical system, generally designated 100, according to an exemplary embodiment. The surgical system 100 includes a console 102 and a vitrectomy probe handpiece 112. The console 102 may include an associated display screen 104 configured to show data relating to system operation and performance during a vitrectomy surgical procedure. In some embodiments, the console 102 may be mobile, and may include wheels to facilitate movement about an operating room as necessary or as desired. In an alternative embodiment, the console 102 may not include wheels. The console 102 may be referred to as a “base housing” and may include a plurality of subsystems that cooperate to enable a surgeon to perform a variety of surgical procedures, such as ophthalmic surgical procedures. The vitrectomy probe handpiece 112 may attach to the console 102 by one or more connection conduits and may form a part of the surgical system 100. Embodiments may include various conduits or conduits to supply high and low fluid pressures, such as air pressure, to provide electrical powers, and other control signals. Two exemplary connection conduits 103A and 103B are illustrated in the exemplary implementation of FIG. 1. In some implementations, the conduits 103A and/or 103B are formed of lengths of tubing forming fluid conduits that convey fluids, such as air, saline, removed vitreous, substance, or others, between the handpiece 112 and one or more subsystems of the console 102. In some embodiments, the connection conduits 103A and/or 103B may each include a plurality of lumens enabling multiple fluids to be conveyed to or from the handpiece 112. In alternative embodiments, the connection conduits 103A and/or 103B may be electrical cables, supplying power and or control signals to and from the handpiece 112. For example, the handpiece 112 may include a switch or other control mechanism (e.g., a button, slider, or roller) to direct the substance to be injected. In some embodiments, the command to inject the substance may be a command to activate a reflux process, thereby reversing flow of fluid. The handpiece 112 may form part of a vitrectomy subsystem described herein.

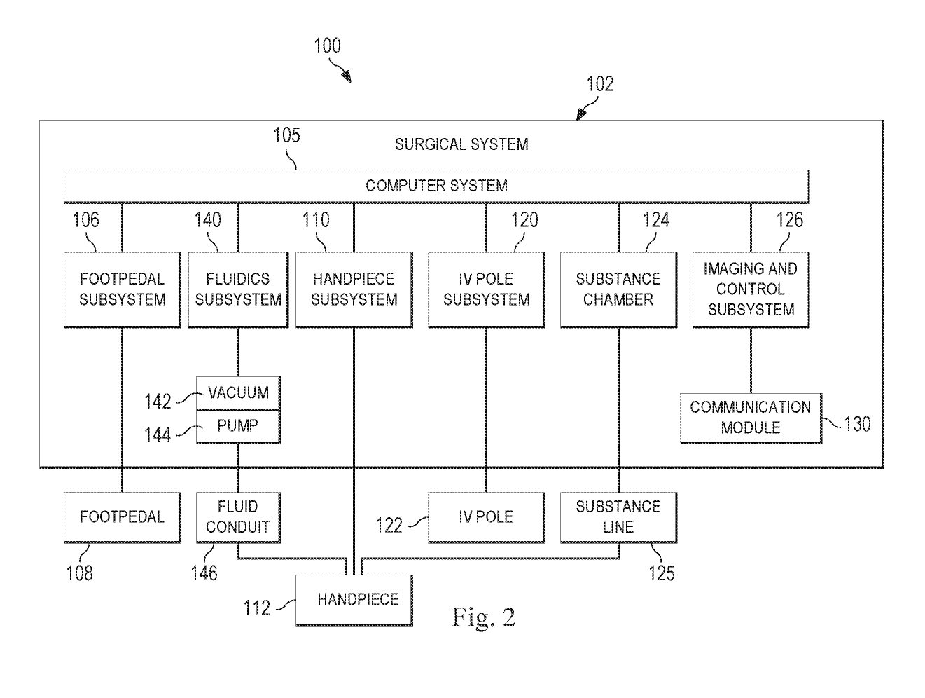

FIG. 2 is a block diagram of the surgical system 100 including the console 102 and several subsystems thereof. The console 102 includes a computer subsystem 105, the display screen 104 (FIG. 1), and a number of subsystems that are used together to perform ocular surgical procedures, such as emulsification or vitrectomy surgical procedures, for example. The computer subsystem 105 may operate or control the subsystems according to instructions to provide proper operation of the subsystem. The computer subsystem 105 may include one or more processing devices, such as a central processing unit or a central processor or a microcontroller, and an information and data storage system. The data storage system may include one or more types of memory, such as RAM (random access memory), ROM (read-only memory), flash memory, a disk-based hard drive, and/or a solid-state hard drive. The processing devices and storage system may communicate over a bus, which may also permit communication with and between one or more of the plurality of subsystems of the surgical system 100.

In the exemplary implementation of FIG. 2, the subsystems include a footpedal subsystem 106 including, for example, a footpedal 108, a fluidics subsystem 140 including an aspiration vacuum 142 and an irrigation pump 144 that connect to fluid conduit 146, which may be tubing expending between the console 102 and the handpiece 112. The surgical system 100 includes a handpiece subsystem 110 including the handpiece 112 and an intravenous (IV) pole subsystem 120 including a motorized IV pole 122. The handpiece subsystem 110 may receive and/or encode signals to and from the handpiece 112 for communication between the handpiece 112 and the computer subsystem 105 to enable the surgeon to use the handpiece 112 to control different subsystems included in the surgical system 100. Some embodiments of the console 102 may include a substance chamber 124 and a substance conduit 125 to conduct substance-containing fluid (e.g., a drug, retina patch, dye, etc.) from the substance chamber 124 to the handpiece 112. The surgical system 100 may further include an imaging and control subsystem 126 including a communication module 130. Other subsystems or tools may be included additionally or alternatively in other embodiments. For example, some embodiments may include a fiber optic illumination subsystem to provide for illumination within a body cavity, such as the vitreous chamber or anterior chamber of an eye. To optimize performance of the different subsystems during surgery, their operating parameters differ according to, for example, the particular procedure being performed, the different stages of the procedure, the surgeon's personal preferences and commands, whether the procedure is being performed in the anterior or posterior portion of the patient's eye, and so on.

The different subsystems in the console 102 comprise control circuits for the operation and control of the respective microsurgical instruments or instrument components. The computer subsystem 105 governs the interactions and relationships between the different subsystems to properly perform an ocular surgical procedure and to properly communicate information to the operator of the surgical system 100 through the display 104 and/or through a coupled microscope or wearable computing device. In some implementations, the processing devices of the computer subsystem 105 are preprogrammed with instructions for controlling the subsystems to carry out a surgical procedure, such as an emulsification procedure or a vitrectomy, for example.

In addition, the console 102 may include one or more input devices that permit a user to make selections within a limited scope to control or modify the preprogrammed relationships between different subsystems. In this embodiment, input devices may be incorporated into the console and may include the footpedal 108, a touch screen device responsive to selections made directly on the screen, a standard computer keyboard, a standard pointing device, such as a mouse or trackball, buttons, knobs, or other input devices are also contemplated. For example, the handpiece 112 may include one or more switches, knobs, touch-sensors, sliders, or other input devices, to enable a user to use the handpiece 112 as an input device as well. Using the input devices, a surgeon, scientist, or other user may select or adjust parameters that affect the relationships between the different subsystems of the console 102. For example, a surgeon may trigger injection of a substance from the substance chamber 124 through the handpiece 112 into the patient's eye. Additionally, a surgeon may change one or more parameters for the operation of the handpiece 112, such as aspiration or reflux parameters or an oscillation parameter of the vitreous cutting mechanism. Accordingly, based on a user input, a user may change or adjust the relationships from those that were coded into the console by the system programmers.

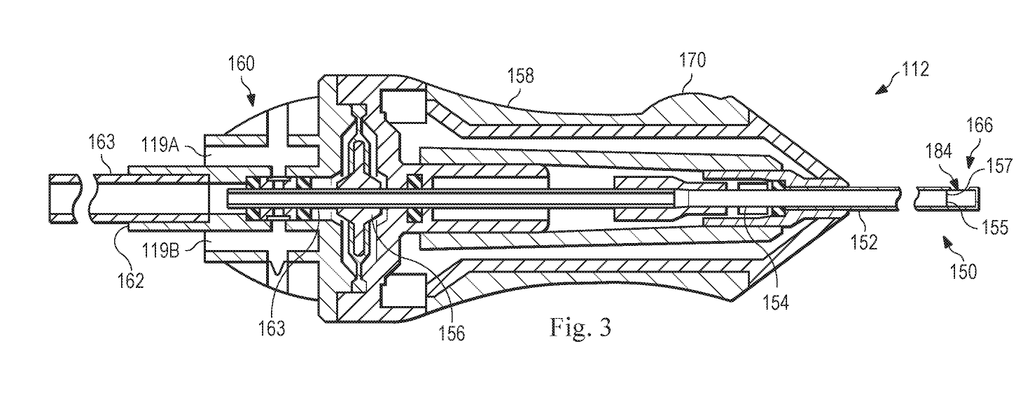

Because the handpiece 112 is configured to inject a substance, the surgeon may be able to better visualize aspects of the surgical operations performed by or near by the handpiece 112, without requiring two incisions and without requiring the manipulation and handling of two separate devices within the small confines of the eye or in another cavity or area of the patient. In some embodiments, the handpiece 112 may be charged or primed prior for use, and then again during use by removing the handpiece 112 from the eye and inserting it into a substance chamber, such as the substance chamber 124 or another substance chamber. In this state, the user may activate the vacuum 142 to pull an amount of substance into the distal tip of the handpiece 112. For example, the surgeon may push a switch on the handpiece 112 to activate the vacuum 142. The switch may activate the vacuum 142 for a predetermined amount of time at a predetermined pressure to draw up a specific amount of substance for injection. Thereafter, the surgeon may replace the distal tip of the handpiece 112 into the eye of the patient. The surgeon may then activate the pump 144 to push the substance into the eye of the patient. In some implementations, the surgeon may activate a control mechanism on the handpiece 112, on the footpedal 108, or the console 102 in order to activate the pump 144 to inject the substance at the desired location. In the example implementation shown in FIG. 3, the illustrated handpiece 112 includes a control mechanism 170, such as a control wheel or selector wheel that may be manipulated by a finger or thumb of the surgeon during a procedure. Other control mechanisms may be included in addition to the selection wheel or other control mechanism 170, as illustrated, or in place thereof. Such control mechanisms may include buttons, switches, or other control mechanisms.

FIG. 3 shows a partial cross-sectional illustration of an exemplary vitrectomy probe that may correspond with the handpiece 112 shown in FIGS. 1 and 2. In this example, the handpiece 112 may be a pneumatically-driven probe that operates by receiving pneumatic pressure alternating through first and second ports 119A and 119B. The handpiece 112 includes as its basic components a vitrectomy cutter 150 comprising an outer cutting tube 152, an inner cutting tube 154 shown in a non-cross-sectional side view, and a probe actuator or motor shown here as a reciprocating air driven diaphragm 156, all partially enclosed by a housing 158. The housing 158 includes an end piece 160 at the handpiece proximal end with the first and second air supply ports 119A and 119B and a port 162 to provide aspiration of liquid and tissue materials from the cutter 150. The port 162 may also provide for the reflux of fluid, such as a fluidic substance (e.g., a drug, retina patch, dye, etc.), out through the cutter 150. As illustrated in FIG. 3, a supply conduit or fluid conduit 163 is coupled to the handpiece 112 at the port 162. In some embodiments, the port 162 may include a plurality of ports or a manifold to couple multiple connection conduits or multiple lumens of a single connection conduit that may be included in some embodiments of the connection conduit 163. Accordingly, multiple fluids may be utilized by the handpiece 112 in connection with the fluidics subsystem 140 (FIG. 2).

In operation, pneumatic pressure is directed alternately from the handpiece subsystem 110 to the first and second ports 119A and 119B to operate the handpiece 112. An on-off pneumatic driver within the handpiece subsystem 110 alternates between its two positions very rapidly to alternatingly provide pneumatic pressure to the first and second ports 119A and 119B. Although shown with a single actuator or motor, other embodiments include two probe actuators or motors, one associated with each of the two ports 119A and 119B. Embodiments of the handpiece 112 may be powered by means other than a pneumatic actuator or actuators. For example, the inner cutting tube 154 may be driven by electromagnetic actuators. In other embodiments, the probe actuator may include a piston motor in place of a diaphragm. In such embodiments, the cutter 150 is arranged so that movement of the piston also moves the inner cutting tube 154 of the cutter 150 relative to the outer cutting tube 152. Yet other embodiments include other types of pneumatic or electric motors that drive the inner cutting tube 154, as will be recognized by those skilled in the art.

FIG. 3 illustrates that the cutter 150 extends from the housing 158 and includes a distal end 166. The outer cutting tube 152 and the inner cutting tube 154 may both be cylindrical tubes or elongate members with a hollow bore or lumen therein. The outer cutting tube 152 has a tissue-receiving outer port 184; the inner cutting tube 154 has an open distal end 155 including a cutting edge that defines an inner cutting port 157. Generally, the inner cutting tube 154 oscillates within the outer cutting tube 152 as driven by the probe actuator. The inner cutting port 157 disposed at the distal tip of the inner cutting tube 154 cuts any vitreous material which may have been aspirated into the tissue-receiving outer port 184 of the outer cutting tube 152. The vitreous may thereafter be aspirated away by the vacuum 142 (FIG. 2) through the distal end 155 of the inner cutting tube 154 and through the lumen extending therethrough. Venting the pressure at the first port 119A and increasing the pressure at the second port 119B moves the diaphragm 156 proximally, allowing more vitreous to enter the lumen of the outer cutting tube 152 through the port 184. If a command is received to inject a substance or a valve system is mechanically adjusted to inject the substance, the substance may move through at least a portion of the inner cutting tube 154 before exiting the port 184 at the site of interest.

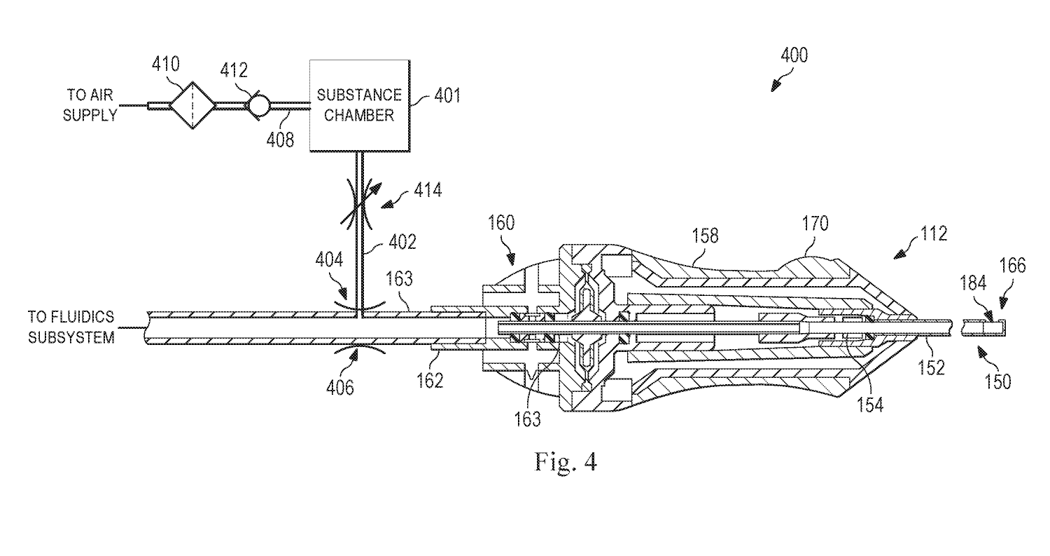

Referring now to FIG. 4, shown therein is an illustration of a tissue removal system 400, according to some embodiments of the present disclosure. FIG. 4 shows an embodiment of the handpiece 112 in cross-section, including the outer cutting tube 152 and the inner cutting tube 154 and the fluid conduit 163, which couples the handpiece 112 to the fluidics subsystem 140 (FIG. 2). FIG. 4 also depicts a substance chamber 401 that is coupled to the handpiece 112. As illustrated in FIG. 4, the substance chamber 401 is coupled to the handpiece 112 by the fluid conduit 163 and an additional fluid conduit 402. The fluid conduit 402 couples the substance chamber 401 to the fluid conduit 163 at a connection site 404. As shown in FIG. 4 the connection site 404 is a location of a venturi 406 formed in the fluid conduit 163. The venturi 406 is a constriction or constricted region within the fluid conduit 163. The venturi 406 affects the pressure within the fluid conduit 163 such that, during reflux, substance is pulled out of the substance chamber 401 through the fluid conduit 402 and the fluid conduit 163, eventually exiting the handpiece 112 at the port 184. When a volume of substance is removed from the substance chamber 401, another fluid, such as air, may pass through yet another fluid conduit 408 to occupy a corresponding volume within the substance chamber 401. Positioned along the fluid conduit 408 are a filter 410 and a check valve 412. The filter 410 may be an air filter or other filter that filters air or another fluid entering the substance chamber 401 during operation. The check valve 412 may be configured to allow air to enter into the substance chamber 401 through the fluid conduit 408 and to prevent air or substance from exiting the substance chamber 401 through the fluid conduit 408.

FIG. 4 also illustrates a variable valve 414 positioned along the fluid conduit 402 to enable control of an injection process by the surgeon or user of the handpiece 112. For example, the valve 414 may be controllable in response to user manipulations of a valve control mechanism such as the control mechanism 170 on the handpiece 112. In some embodiments, the control mechanism 170 may include an encoder that generates electronic signals that are transmitted to the console 102 and the handpiece subsystem 110 therein (FIG. 2). The computer subsystem 105 may receive control signals from the handpiece subsystem 110 and communicate the signals to the fluidics subsystem 140 which may be used to electrically control the state (open, closed, or another position therebetween) of the variable valve 414.

In some embodiments of the tissue removal system 400, the substance chamber 401 is included within the housing 158. In such embodiments, the fluid conduit 402 may connect to the lumen of the inner cutting tube 154 without connecting directly to the fluid conduit 163 at the connection site 404. In other embodiments, the housing 158 may include a conforming recess configured to receive a cartridge including the substance chamber 401. When the cartridge is inserted into the conforming recess, an opening may be formed in the cartridge to form a fluid connection to allow fluid to be pushed from the substance chamber 401 inside the cartridge out through the port 184. The handpiece 112 may include a conduit extending from the cartridge opening to the proximal end of the inner cutting tube 154. For example, a conduit may introduce substance into the fluid pathway between the fluid conduit 163 and the proximal end of the inner cutting tube 154 within the port 162.

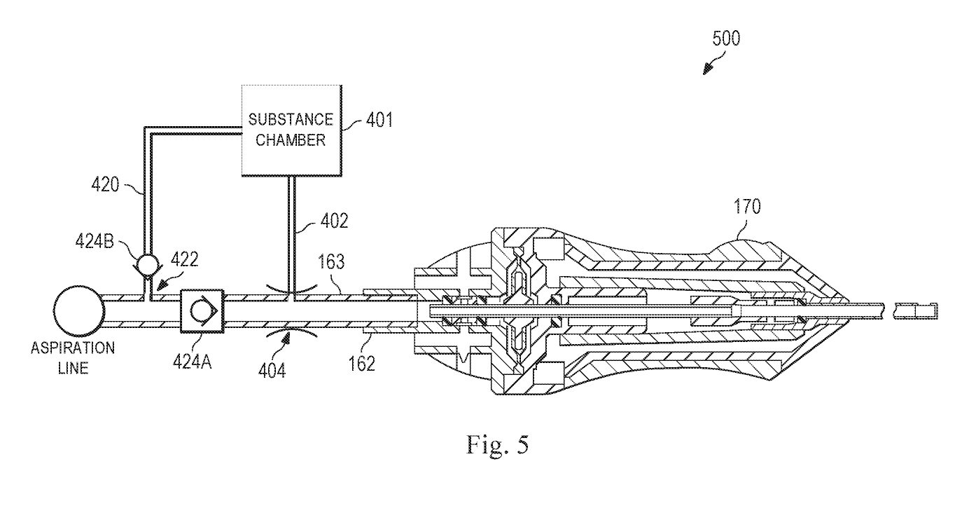

Referring now to FIG. 5, shown therein is an illustration of an embodiment of a tissue removal system 500, according to some embodiments of the present disclosure. The tissue removal system 500 as illustrated in FIG. 5 has many features in common with the tissue removal system 400 illustrated in FIG. 4. For example, as shown in FIG. 5, the tissue removal system 500 includes a handpiece 112 with a vitreous cutter 150 at a distal end thereof. A proximal end of the handpiece 112 is coupled to a fluid conduit 163 at a port 162 of the handpiece 112. The system includes a substance chamber 401 that is coupled to the handpiece 112.

As illustrated in FIG. 5, the substance chamber 401 is coupled to the handpiece 112 by an additional fluid conduit 402 and a portion of the fluid conduit 163. The additional fluid conduit 402 is connected to the fluid conduit 163 at a connection site 404. The connection site 404 may include an opening in a wall of the fluid conduit 163 such that a lumen of the fluid conduit 402 is in fluid communication with a lumen of the fluid conduit 163. The substance chamber 401 is also coupled to the fluid conduit 163 by a fluid conduit 420. The fluid conduit 420 connects the substance chamber 401 to the fluid conduit 163 at a connection site 422. The connection site 422 has a more proximal position than the connection site 404. The embodiment of the tissue removal system 500 illustrated in FIG. 5 includes a plurality of check valves. A first check valve 424A is positioned along the fluid conduit 163. The check valve 424A is configured to permit aspiration or suction of material from the vitreous cutter 150 of the handpiece 112 through the fluid conduit 163 to the fluidics subsystem 140 of FIG. 2. The check valve 424A prevents fluid from passing from the fluidics subsystem 140 to the handpiece 112 along the portion of the fluid conduit 163 between the connection sites 422 and 404. A check valve 424B is positioned along the fluid conduit 420 between the connection site 422 and the substance chamber 401. The check valve 424B is configured to prevent substance from flowing from the substance chamber 401 to the fluid conduit 163 by way of the connection site 422. The check valve 424B permits fluid to travel from the fluidics subsystem 140 (FIG. 2) to the substance chamber 401. As fluid enters the substance chamber 401 through the fluid conduit 420, substance exits the substance chamber 401 through the fluid conduit 402 and into the fluid conduit 163 at the connection site 404. Because of the check valve 424A, the substance is directed into the handpiece 112 and out through the port 184 of the vitreous cutter 150.

Accordingly, a surgeon may use a control mechanism 170 on the handpiece 112 to signal to the fluidics subsystem 140 to activate the pump 144. The pump 144 responsively displaces fluid through the fluid conduit 163, which is routed into the substance chamber 401 causing substance to be ejected from the handpiece 112. In some embodiments, the footpedal 108 (FIG. 2) is used to activate the pump 144 to inject substance into a treatment site, such as a vitreous chamber. When the vacuum 142 (FIG. 2) is activated, the fluidics subsystem 140 may be used along with the vitreous cutter 150 to remove vitreous from the vitreous chamber of the patient's eye as normal. In other embodiments, another tissue may be removed from another body cavity. The handpiece 112 may be operated without reflux, in an aspiration mode only, and no substance will be injected.

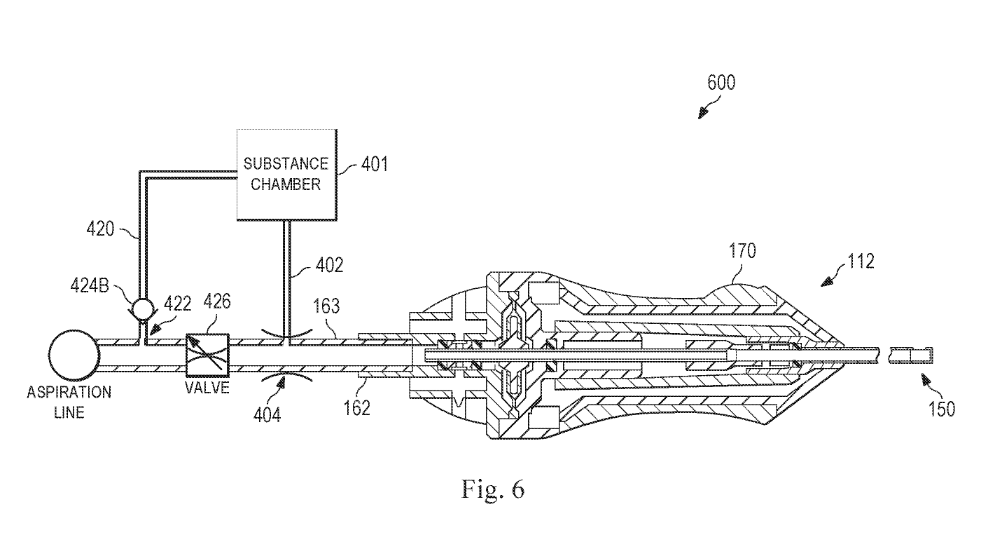

Referring now to FIG. 6, shown therein is an illustration of an embodiment of a tissue removal system 600, according to some embodiments of the present disclosure. The illustrated tissue removal system of FIG. 6 shares many features with the systems 400 and 500 illustrated in FIGS. 4 and 5, respectively, and described herein. The substance chamber 401 of FIG. 6 is coupled to the handpiece 112 by the fluid conduit 402 and fluid conduit 420, which connect to the fluid conduit 163 at connection sites 422 and 404, respectively. In other embodiments, the substance chamber 401 may be included inside the handpiece 112 and may be coupled directly to the port 162 (e.g., the fluid conduit 402 may couple directly to the port 162, without physically contacting the fluid conduit 163). As in FIG. 5, the fluid conduit 420 includes a check valve 424B allowing for the flow of fluid from the fluid conduit 163 to the substance chamber 401 but stopping flow of fluid from the substance chamber 401 to the fluid conduit 163 at connection site 422. Instead, the substance is directed to the handpiece 112 through the fluid conduit 402. From the fluid conduit 402, the substance may flow through the fluid conduit 163 as illustrated or, in other embodiments, directly to the port 162. From the port 162, the substance travels through the inner cutting tube 154 and out through the port 184 (FIG. 2).

Instead of including the check valve 242A (FIG. 5) the system 600 illustrated in FIG. 6 includes a variable valve 426. The variable valve 426 is mechanically, electronically, or pneumatically controllable by the operator of the system. For example, a surgeon may use the control mechanism 170 to aspirate cut tissue away from a surgical site by setting the state of the variable valve 426 to be open and by sending controls to the console 102 to activate the vacuum 142 (FIG. 2). In order to inject substance, the surgeon may use the control mechanism 170 to set the state of the variable valve 426 to the closed state. The surgeon may then control the console 102 to activate the pump 144 causing fluid to flow within the fluid conduit 163 toward the handpiece 112. The flow is redirected by the closed variable valve 426 to pass through the check valve 424B, through the remainder of the fluid conduit 420 and into the substance chamber 401. In the substance chamber 401, the entering fluid may displace the substance out through the fluid conduit 402. As illustrated, the substance passes into the distal portion of the fluid conduit 163 and through the handpiece 112. In other embodiments, the substance passes through the fluid conduit 402, or another conduit, directly into the port 162 (FIG. 2) and then out through the handpiece 112 into the surgical site. The substance attaches to the vitreous, making the vitreous easier to visualize for removal.

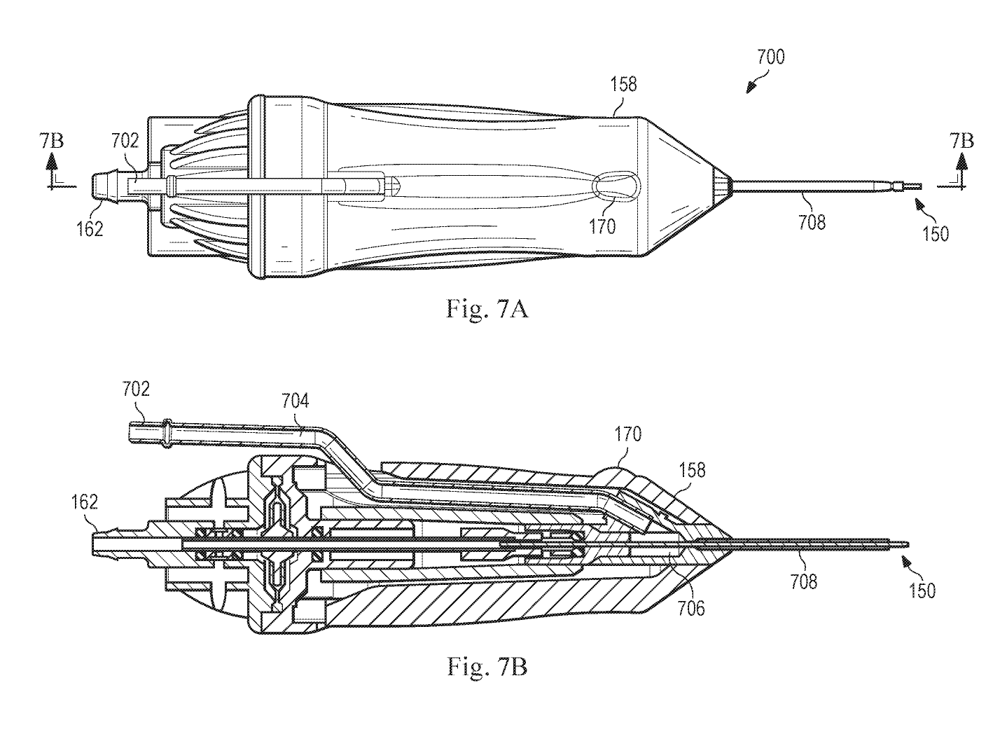

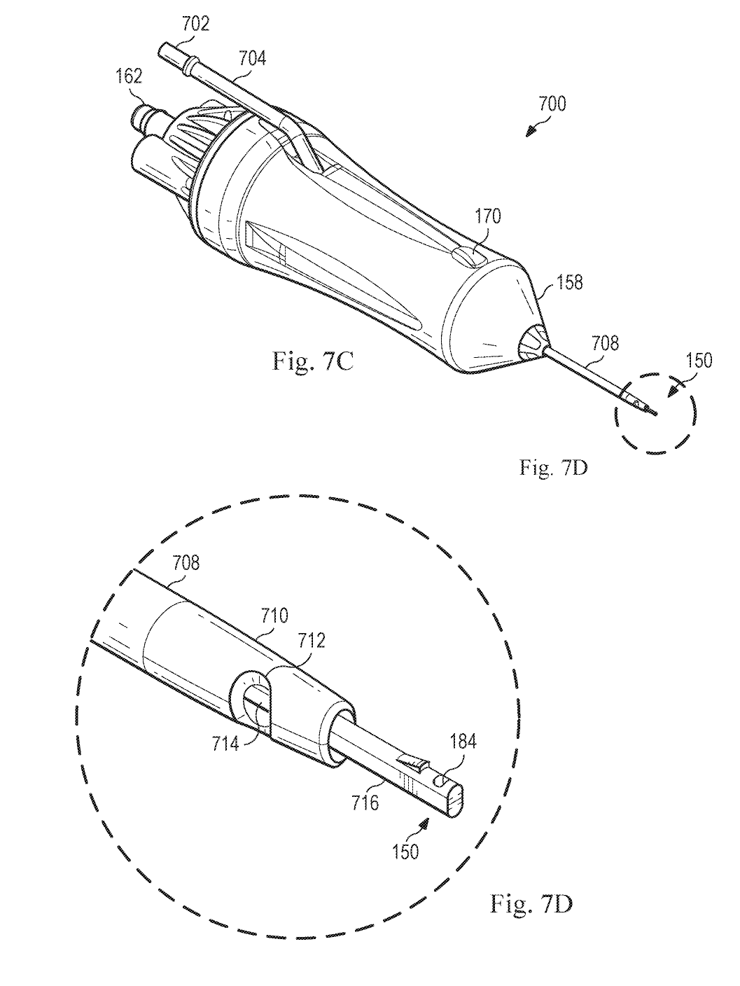

Referring now the FIGS. 7A, 7B, 7C, and 7D, shown therein are illustrations of a handpiece 700 for use in a tissue removal surgical system like the system 100 of FIG. 1 and/or the systems 400, 500, and 600 of FIGS. 4, 5, and 6, respectively. The handpiece 700 may be understood as an embodiment of the handpiece 112. FIG. 7A is a top view illustration, showing an outside of the handpiece 700. The handpiece 700 includes many of the features describe herein as associated with the handpiece 112. For example, the handpiece 700 includes a vitrectomy cutter 150 having an inner cutting tube 714 and an outer cutting tube 716 (seen in detail in FIG. 7D). The vitrectomy cutter 150 is present at a distal end of the elongate member protruding from the handpiece 700. The handpiece 700 includes a port 162 that has an outer surface with a barb to securely hold a distal end of the fluid conduit and create a seal therebetween, so that the fluid conduit 163 couples the handpiece 700 to the fluidics subsystem 140 of FIG. 2. An inner chamber of the port 162 is in fluid communication with a lumen that extends through to the distal end of the vitreous cutter 150.

The handpiece 700 further includes a substance port 702, which is coupled to a fluid conduit 704 that extends within the housing 158 for the distal end thereof. A cross-sectional view of the fluid conduit 704 is depicted in FIG. 7B, which is a cross-sectional view of the handpiece 700 as viewed along the conduit A-A of FIG. 7A. Within a distal end of the housing 158, a lumen of the fluid conduit 174 may connect to a substance distribution chamber 706. The chamber 706 may couple to an elongate tubular member 708, which may extend around the inner cutting tube 714 of the vitreous cutter 150. The lumen of the elongate member 708 may include an inner diameter that is greater than an outer diameter of the inner cutting tube 714 of the vitreous cutter 150. As such, the gap is present between the inner surface of the elongate member 708 and the outer surface of the inner cutting tube 714. The chamber 706 may direct fluidic substance introduced into the handpiece 700, via port 702 and the fluid conduit 704, into the gap and toward the vitreous cutter 150. The substance may flow through the gap toward the distal end of the vitreous cutter 150, from which it may be injected into the surgical site.

FIG. 7C is a perspective view of the handpiece 700 and includes a region of interest B, which is presented in more detail in FIG. 7D. FIG. 7D shows an embodiment of the vitreous cutter 150 and a distal portion of the elongate member 708. As illustrated in FIG. 7D, the example elongate member 708 may include a tapered section 710 at a distal end thereof. The tapered section 710 may include a port 712 that directs substance radially away from a central axis of the elongate member 708. In the illustrated implementation, a portion of the inner cutting tube 714 can be seen in the port 712. In such implementations, a proximal end of the outer cutting tube 716 may be fixed within the distal end of the tapered section 710 and extend distally therefrom. In addition, a gap between the inner surface of the elongate tubular member 708 and the outer surface of the inner cutting tube 714. Accordingly, the outer cutting tube 716 may not extend the full length of the elongate member 708 but may be affixed to a distal end of the elongate member 708, in some implementations. In other embodiments, the outer cutting tube extends along the elongate member 708 such that the port 712 exposes the outer cutting tube 716. In some embodiments, the connection between the outer cutting tube 716 and the elongate member 708 is a sealed connection, such that all substance flowing through the elongate member 708 is directed through the port 712. Some embodiments may include multiple ports like the port 712, which may be distributed circumferentially to direct substance all around the distal end of the elongate member 708. In some implementations, the port 712 is aligned with the outer port 184. Additionally, some embodiments may include one or more ports or openings at the distal end of the tapered section 710 such that at least a portion of the fluid is directed along the central axis of the elongate member 708 in the direction of the port 184.

While the illustrated embodiment of FIG. 7D shows the outer cutting tube 716 extending from the distal end of the tapered section 710, other embodiments may direct a substance or other fluid, such as a bioactive fluid, into the eye. Still other embodiments may direct the substance through a gap present between the outer surface of the outer cutting tube 716 and the inner surface of the elongate member 708. The user may manipulate the control mechanism 170, the footpedal 108, or another input device, to cause the console to activate the pump 144 (FIG. 2) to inject the substance or other fluid into the surgical site to improve visualization of tissue.

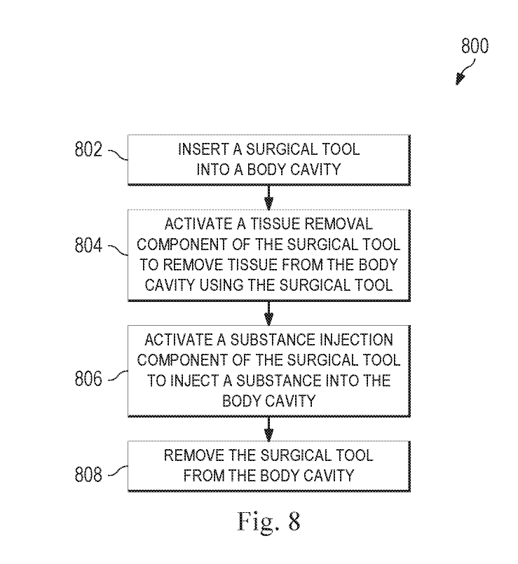

Referring now to FIG. 8, shown therein is a method 800 of performing a surgical procedure to remove tissue from a surgical site. As illustrated in FIG. 8, the method 800 includes a plurality of enumerated steps or operations. Embodiments of the method 800 may include additional operations before, after, in between, or as part of the enumerated operations. Additionally, some embodiments of the method 800 may not include all of the illustrated operations. One or more of the operations may be provided as instructions, stored on a computer readable media, that may be executed by a computer system like the computer subsystem 105 of FIG. 2. As shown in FIG. 8, an embodiment of the method 800 may begin at 802 when a surgical tool is inserted through the body cavity. For example, a surgeon may insert a vitreous cutter, like the vitreous cutter 150 of the handpiece 112 described herein, through a trocar cannula that provides access to the vitreous chamber of an eye.

At 804, the tissue removal component of the surgical tool may be activated to remove tissue from the body cavity using a surgical tool. For example, the computer subsystem 105 may receive a command from the handpiece 112 or the handpiece 700 to activate the vitreous cutter 150 of the handpiece 112 or the handpiece 700. At 806, substance injection may be activated to inject a substance into the body cavity. For example, the command may be received by the computer subsystem 105 from the handpiece 112 or handpiece 700 to activate the pump 144 of the fluidics subsystem 140 (FIG. 2). Additionally, the command may direct the computer subsystem 105 (FIG. 2) to adjust one or more electronically-controlled valves of the tissue removal system. For example, the computer subsystem 105 may receive a command from the control mechanism 170 to control the variable valve 414 (FIG. 4) or the variable valve 426 (FIG. 6). By controlling one or more variable valves, a fluidic substance may be ejected from or near the distal end of the vitreous cutter 150 into the tissue surrounding the vitreous cutter 150. The substance may be a drug, a retina patch or a dye (e.g., used to increase visibility of the tissue, particularly when the tissue is transparent or translucent, as is vitreous).

After the substance has been injected at 808, some implementations of the method 800 may return to 804 at which the tissue removal component is activated again. For example, a surgeon may use an embodiment of the handpiece described herein to remove the vitreous in the patient. The surgeon may then use a device to activate a substance injection process to improve visibility of a portion of the main vitreous. The surgeon may thereafter reactivate or request reactivation of the vitreous cutter to remove additional vitreous. At 808, the surgical tool is removed from the body cavity. For example, after removing the desired amount of vitreous, the surgeon may remove the vitreous cutter 150 from the vitreous chamber of the eye of the patient. Subsequent operations may be performed thereafter depending on the condition to be treated.

Embodiments of the present disclosure may include methods of performing a surgical procedure using surgical device, like the handpieces 112 and 700 described herein. In an ophthalmic example, a surgeon may make an incision in the eye of a patient. The surgeon may then insert the vitrectomy cutter 150 through the incision. In some embodiments, a trocar cannula may be positioned in the incision and the cutter 150 may be advanced therethrough. The surgeon may then inject a desired amount of substance (e.g., a dye to improve visibility of the vitreous) into the eye. The dye may enable to surgeon to better see the vitreous in order to remove it more effectively and safely. Other substances are also contemplated (e.g., a drug, retina patch, etc).

Through use of principles described herein, a user can deliver a substance, such as a drug, retina patch, dye, etc. into the eye as needed. In the case of a dye, the user may have a better experience when viewing tissue at the surgical site. Specifically, the user may be better able to visualize clear vitreous or other transparent or translucent tissue by introducing a dye that adheres to the tissue. The tissue may then be more readily removed. The integrated substance injector of tissue removal systems described herein may simplify the surgical procedure.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A tissue removal surgical system comprising:
a tissue removal handpiece having a tissue cutter disposed at a distal end;
a first fluid conduit coupling a fluidics subsystem to the tissue removal handpiece to permit removal of tissue cut by the tissue cutter of the tissue removal handpiece;
a second fluid conduit coupling a substance chamber to the tissue removal handpiece; and
a control system facilitating selective control of fluid through the tissue removal handpiece to introduce a substance from the substance chamber to a surgical site through a port in the tissue cutter.

2. The tissue removal surgical system of claim 1, wherein the second fluid conduit is connected to the first fluid conduit to convey the substance from the substance chamber through the second fluid conduit and a portion of the first fluid conduit.

3. The tissue removal surgical system of claim 2, wherein the second fluid conduit connects to the first fluid conduit at a constricted region of the first fluid conduit.

4. The tissue removal surgical system of claim 1, further comprising a third fluid conduit coupled to the substance chamber, the third fluid conduit permitting a fluid other than the substance to enter into the substance chamber.

5. The tissue removal surgical system of claim 4, further comprising an air filter coupled to the third fluid conduit to filter air entering the substance chamber during injection of the substance from the substance chamber into the surgical site.

6. The tissue removal surgical system of claim 5, further comprising a valve between the substance chamber and the tissue removal handpiece, and wherein the control system comprises a valve control mechanism to control the valve, the valve control mechanism being disposed on the tissue removal handpiece to enable a user to control injection of the substance from the substance chamber to the surgical site.

7. The tissue removal surgical system of claim 6, wherein the valve control mechanism comprises a switch, button, slider, or roller on an exterior of the handpiece and accessible to the user for controlling the injection of the substance.

8. The tissue removal surgical system of claim 1, further comprising:
a third fluid conduit that couples the substance chamber to the first fluid conduit at a connection site along the first fluid conduit; and
a check valve disposed along the third fluid conduit, the check valve permitting fluid to pass from a first portion of the first fluid conduit into the substance chamber.

9. The tissue removal surgical system of claim 8, further comprising an additional check valve disposed on the first fluid conduit between the connection site and the tissue removal handpiece, the additional check valve being disposed to pass material through the first fluid conduit from the surgical site to the fluidics subsystem.

10. The tissue removal surgical system of claim 8, further comprising a variable valve disposed on the first fluid conduit between the connection site and the tissue removal handpiece.

11. A surgical instrument for use in an ophthalmic procedure, the surgical instrument comprising:
an elongate tubular member having a distal end for insertion through eye tissue into the vitreous chamber of an eye, the elongate tubular member having an inner lumen extending therethrough, a portion of the elongate tubular member extending within a housing;
a first fluid conduit to couple the housing to a fluidics subsystem to aspirate vitreous from the vitreous chamber of the eye, the first fluid conduit in fluid communication with the lumen of the elongate tubular member;
a substance chamber coupled to the first fluid conduit to provide a substance into the vitreous chamber of the eye, wherein the substance chamber is coupled to the first fluid conduit at a first connection site via a second fluid conduit and at a second connection site via a third fluid conduit;
a first valve positioned along the second fluid conduit; and
a second valve positioned along the first fluid conduit between the first connection site and the second connection site.

12. The surgical instrument of claim 11, wherein the substance chamber is positioned within the housing.

13. The surgical instrument of claim 11, wherein the substance chamber is a cartridge configured to mate with a cartridge-receiving portion disposed on an exterior surface of the housing.

14. The surgical instrument of claim 11, wherein the first valve and the second valve are check valves.

15. The surgical instrument of claim 11, wherein the first valve is a check valve and the second valve is a variable valve that is controllable by a user when the user manipulates a valve control mechanism disposed on the housing.

16. The surgical instrument of claim 11, further comprising a cutting tube disposed within the distal end of the elongate tubular member.

17. A tissue removing surgical system comprising:
a vitrectomy handpiece including:
a housing configured for use in a user's hand during a vitrectomy procedure; and
a vitrectomy cutter protruding from a distal end of the housing, the vitrectomy cutter comprising:
an inner cutting tube having an inner cutting port at a distal tip thereof,
an elongate tubular member having a lumen extending therethrough, the inner cutting tube extending within the lumen of the elongate tubular member; and
an outer cutting tube having an outer port, the outer cutting tube extending from a distal end of the elongate tubular member;
a fluid conduit coupled to the handpiece and coupled to the vitrectomy cutter; and
a substance infusion conduit coupled to the handpiece and coupled to the elongate tubular member.

18. The tissue removing surgical system of claim 17, wherein the fluid conduit couples the handpiece to a fluidics subsystem in a vitrectomy console.

19. The tissue removing surgical system of claim 18, wherein the handpiece further comprises a substance infusion mechanism to provide for a controlled injection of substance into a surgical site.

20. The tissue removing surgical system of claim 17, wherein the handpiece further includes an internal chamber to distribute the substance between the vitrectomy cutter and the elongate tubular member.

21. The tissue removing surgical system of claim 20, wherein the elongate tubular member comprises a substance exit port located proximate the distal end of the elongate tubular member.
